(19) United States
(12) Patent Application Publication
(10) Pub. No.: US 2014/0134597 A1
(43) Pub. Date: May 15, 2014

(54) CELLULAR HEMOGLOBIN A1C QUALITY CONTROLS

(71) Applicant: Bio-Rad Laboratories, Inc., Hercules, CA (US)

(72) Inventors: Alireza Ebrahim, Laguna Niguel, CA (US); Karl De Vore, Coto de Caza, CA (US); Christopher Spates, Laguna Hills, CA (US)

(73) Assignee: Bio-Rad Laboratories, Inc., Hercules, CA (US)

(21) Appl. No.: 14/062,351

(22) Filed: Oct. 24, 2013

Related U.S. Application Data
(60) Provisional application No. 61/726,679, filed on Nov. 15, 2012.

(51) Int. Cl. C12N 5/078

(52) U.S. Cl. CPC C12N 5/0641
USPC 435/2; 435/375

(57) ABSTRACT

Intact erythrocytes with selected and often elevated levels of hemoglobin A1c (HbA1c) for use as quality controls for HbA1c assays and assay instruments are prepared by hypotonic dialysis of erythrocytes from a healthy mammal to permeabilize the erythrocyte membranes, infusion of the permeabilized erythrocytes with hemoglobin A1c, and de-permeabilization of the infused erythrocytes. Quality controls of essentially any level of HbA1c can be prepared in this manner and once prepared will be useful for monitoring the entire assay procedure, including the lysis of the erythrocytes in a typical sample.

### CROSS-REFERENCE TO RELATED PATENT APPLICATIONS

The present application claims benefit of priority to U.S. Provisional Patent Application No. 61/726,679, filed Nov. 15, 2012, which is incorporated by reference.

### BACKGROUND

1. Field of the Invention

This invention resides in the field of quality controls of clinical diagnostic instruments, and particularly of controls for instruments used in measuring levels of hemoglobin A1c in mammalian blood.

2. Description of the Prior Art

Determinations of the level of hemoglobin (Hb) in human blood are widely used for the detection, diagnosis, and monitoring of certain diseases. Anemia and sickle cell disease, for example, cause hemoglobin levels to drop, while polycythemia and erythrocytosis cause them to rise. Glycated forms of hemoglobin are of particular interest, notably in the management of diabetes mellitus. The glycated forms result from the reaction of hemoglobin with the free glucose present in human plasma, and in approximately 80% of all glycated Hb the glucose is joined to Hb at the N-terminal amino group of the HbA beta chain. This form of glycated hemoglobin is known as hemoglobin A1c or HbA1c. The formation of HbA1c is slow but irreversible, and the blood level of HbA1c depends on both the life span of the red blood cells (which averages 120 days) and the blood glucose concentration. Thus, although blood glucose levels fluctuate widely, HbA1c levels do not, with the result that HbA1c is a reliable and therefore favored indicator of blood glucose.

Among clinical methods for the detection and measurement of HbA1c, a variety of methodologies are available, examples of which are ionic-exchange high performance liquid chromatography (HPLC), immunoinhibition turbidimetric techniques, and boronate affinity chromatography. Each of these techniques requires the lysis of the red blood cells (erythrocytes) in the sample, either manually or by automated instrumentation, to release the HbA1c and the cell contents in general for analysis. In conducting these tests, it is important to maintain quality control for assuring precision and accuracy in use of the instrumentation and analytical materials. Quality control materials are in fact useful for a variety of purposes, including serving as reference standards for routine use in determinations and as tools for user training, in addition to providing checks on the condition of all reagents and other materials that are used in the test.

Commercially available quality control materials for many analytes are prepared by adding precise quantities of the analyte, together with stabilizers, antimicrobial agents, and other additives, to a base matrix. Base matrices are often processed human fluids such as human serum or human urine to ensure that the quality control is as sensitive as an actual patient sample to all anticipated analytical variances. Quality controls can be found in either single-analyte or multi-analyte form, and often in bi-level or tri-level configurations to allow test methods to be monitored and challenged at analyte levels above, near, and below the medical decision point for each assay. Many multi-analyte controls have lists of related analytes, for example tumor markers, or analytes measured by one type of detection technology, such as, for example, photometry or reflectance photometry. Regardless of what they are designed for and how they are configured, however, quality controls must have lot-to-lot reproducibility and be both cost effective and stable.

For HbA1c, a variety of controls representing both normal and abnormal levels are available. Almost all are in the form of lyophilized protein powders or hemolyzed liquid solutions. An ideal quality control is one that monitors the entire testing process, however, including any sample pretreatment steps, which in the case of HbA1c includes lysis. Cellular controls, i.e., those that are intact RBCs, have indeed been used, although they have limitations as well. Those that are prepared from screened blood units will have HbA1c concentrations that do not exceed the concentrations found in the body, even if drawn from individuals with abnormally high concentrations. The upper limit of HbA1c from these sources is approximately 9%, making the controls inadequate for monitoring the packaged assays that are available from commercial suppliers, whose measuring ranges extend as high as 16%. Even for cellular controls at 9% HbA1c, large quantities of RBC units must be screened to achieve even a modest amount of units that will be acceptable for processing as controls. This is illustrated by the disclosure in Ryan et al. U.S. Pat. No. 7,361,513 B2 (issued Apr. 22, 2008), which describes the preparation of cellular HbA1c controls at both normal levels and abnormal (diabetic) levels. To obtain units suitable as the raw materials for Level II (abnormal) controls, Ryan et al. screened 1400 units from donors weighing 180 pounds or higher, to select only those that had at least 9% HbA1c and normal levels of HbA1a, HbAb, and HbA1f, that lacked abnormal hemoglobin units such as HbS and HbC, that lacked visible clots, and that lacked a significant amount of weak cells (indicative of abnormal levels of hemolysis). Only 37 of the 1400 units met these requirements, indicating a qualification rate of only 2.6% (U.S. Pat. No. 7,361,513 B2, column 7, lines 9-23).

An alternative to screening large quantities of RBC units to obtain units at high target levels is direct glycation, methods of which are also disclosed by Ryan et al. U.S. Pat. No. 7,361,513. These methods use RBCs containing approximately 6% HbA1c, and involve incubation of these RBCs with glucose in a glucose-rich (1-6% by weight) isotonic solution at 6° C. and pH 6-8. A disadvantage of this procedure is that it requires a long incubation time. As described by Ryan et al., normal RBC units that were incubated in a 3.15% glucose solution for fifty days at 6° C. underwent only a 2.6% increase in HbA1c, indicating an average growth rate of only 1% every twenty days (U.S. Pat. No. 7,361,513 B2, column 7, lines 9-23). A further disadvantage is that incubation of RBCs in glucose-containing solutions can result in poor commutability due to non-specific and uncontrolled glycation. For these reasons, the direct glycation of RBCs is not well suited to commercial manufacturing.

### SUMMARY

It has now been discovered that cellular HbA1c controls in which the HbA1c is encapsulated in intact mammalian RBCs (erythrocytes) can be prepared in a consistent and economical manner and without many of the limitations of the prior art by dialyzing RBCs in their native condition against a hypotonic solution under conditions that will result in permeabilization of the RBC cell membranes, contacting the RBCs with permeabilized membranes with a solution of hemoglobin A1c at a selected concentration to equilibrate the RBCs to the solution and thereby infuse the RBCs with hemoglobin A1c from the solution, and then contacting the equilibrated RBCs with a non-hypotonic solution under conditions resulting in the de-permeabilization of the cell membranes, i.e., the sealing of the cells with the encapsulated hemoglobin A1c. The resulting RBCs contain hemoglobin A1c at a stabilized level and are thus ready for use as a quality control. At any of various points during the procedure, the RBCs can be fixed, stabilized, or otherwise treated by treatment with an appropriate agent or agents or by appropriate techniques for such treatments. The cellular controls can contain any level of HbA1c, which is controlled by using a contacting solution with an appropriate HbA1c concentration, and the procedure can be varied by including various additional steps and alternative means of performing the steps described above to suit particular needs and to tailor the resulting controls to meet those needs. In certain cases, the procedure will result in novel controls.

In some embodiments, a method of manufacturing a cellular hemoglobin A1c quality control comprising intact mammalian erythrocytes encapsulating hemoglobin A1c is provided. In some embodiments, the method comprises:
(a) dialyzing erythrocytes from a healthy mammal against a hypotonic solution under conditions causing permeabilization of cell membranes of said erythrocytes;
(b) contacting said erythrocytes having said permeabilized membranes with a solution of hemoglobin A1c at a selected concentration to infuse said erythrocytes with hemoglobin A1c from said solution; and
(c) contacting said erythrocytes so infused with a non-hypotonic solution under conditions causing de-permeabilization of said erythrocytes, thereby achieving intact erythrocytes with a stabilized level of encapsulated hemoglobin A1c.

In some embodiments, the selected concentration of hemoglobin A1c is from 1% to 5% by weight. In some embodiments, said selected concentration of hemoglobin A1c is from 5% to 20% by weight.

In some embodiments, the method further comprises fixing said erythrocytes subsequent to step (c) by treating said erythrocytes with an erythrocyte fixing agent.

In some embodiments, the non-hypotonic solution is a hypertonic solution.

In some embodiments, the method further comprises combining said erythrocytes produced in step (c) with intact mammalian erythrocytes from a healthy mammal that have not undergone steps (a), (b), or (c) in a selected proportion to achieve a quality control with an intermediate level of hemoglobin A1c.

In some embodiments, the method further comprises combining said erythrocytes produced in step (c) with intact mammalian erythrocytes from a healthy mammal that have not undergone steps (a), (b), or (c) in a plurality of proportions to achieve a plurality of quality controls at different levels of hemoglobin A1c.

Also provided is a cellular hemoglobin A1c quality control (e.g., comprising a heterologous A1c protein). For example, in some embodiments, the control is prepared by a method as described above or otherwise herein. In some embodiments, the intact mammalian erythrocytes encapsulating hemoglobin A1c are suspended in a diluent having an osmolality of 200 to 400 mOsm/kg. In some embodiments, the stabilized level of hemoglobin A1c is from 1% to 5% by weight. In some embodiments, the stabilized level of hemoglobin A1c is from 5% to 20% by weight.

Further objects, aspects, embodiments, and advantages of the procedure and the controls will be apparent from the description that follows.

### DETAILED DESCRIPTION OF SELECTED EMBODIMENTS

The sources for RBCs to be used in the procedures described herein can be mammals in general, and for quality controls to be used in conjunction with assays on human samples, human RBCs will be the most appropriate. RBCs from healthy source subjects, i.e., RBCs whose hemoglobin and HbA1c levels are normal, or approximately average for disease-free adult subjects, will often be the most convenient. The RBCs can be used without having been screened to select those with particular levels of hemoglobin or HbA1c, and yet can be subjected to preliminary processing in accordance with conventional processing techniques for cleaning and conditioning RBCs prior to any of the assays typically conducted on RBCs, or any of the other uses of RBCs. Such preliminary processing may include filtration to remove leukocytes or other cellular or particulate material present in the source blood, washing of the RBCs to extract them from their native plasma or sera, dilution of the RBCs, or pelletization, or two or more of these processing steps in sequence or combination. The preliminary processing will not however include fixation.

Permeabilization of the RBCs is then achieved by dialysis against a hypotonic solution. Hypotonic dialysis will cause hemoglobin originally residing in the cells to pass out of the cells through the permeabilized membranes, as well as the HbA1c in the surrounding solution to pass into the cell interiors through the same membranes, and these two effects can be achieved either sequentially or simultaneously. In sequential methods, dialysis will begin with a hypotonic solution that contains neither glycated nor non-glycated hemoglobin or that contains a level low enough to cause a substantial majority of the native hemoglobin to leave the cells, and the hypotonic solution will then be exchanged for a second hypotonic solution that contains dissolved HbA1c in a concentration and amount selected to produce the desired HbA1c level in the cells as quality control materials. In simultaneous methods, the native RBCs will be dialyzed directly, i.e., without a preliminary dialysis, against a hypotonic solution that contains the dissolved HbA1c in the selected concentration and amount, and dialysis will be continued for a period of time sufficient to equilibrate the hemoglobin, glycated and non-glycated, originally inside the cells with that in the surrounding solution.

Between the two methods, sequential dialysis offers the advantage of achieving target levels of HbA1c in the cells independently of the initial hemoglobin content of the cells, and thus in many cases, higher HbA1c levels. As one example of a sequential procedure, a pellet of isolated RBCs can be resuspended in a solution of 10 mM HEPES, 140 mM NaCl, and 5 mM glucose at pH 7.4, and dialyzed against a low ionic strength buffer containing 10 mM NaH2PO4, 10 mM NaHCO3, 20 mM glucose, and 4 mM MgCl2, pH 7.4. After 30-60 minutes, the RBCs are further dialyzed against a 16 mM NaH2PO4, pH 7.4 solution containing the HbA1c at the desired concentration for an additional 30-60 min. These procedures may provide optimal results when performed at a temperature of 4° C.

In general, hypotonic dialysis of RBCs can be performed according to methods known in the art. Examples of descriptions of the procedure are found in Ryan et al. U.S. Pat. No. 5,432,089 (Jul. 11, 1995); McHale et al. U.S. Pat. No. 6,812,204 (Nov. 2, 2004); Hyde et al. U.S. Pat. No. 8,211,656 (Jul. 3, 2012); Franco et al. U.S. Pat. No. 4,931,276 (Jun. 5, 1990); Ropars et al. U.S. Pat. No. 4,652,449 (Mar. 24, 1987); DeLoach, JR, “In Vivo Survival of [14C]Sucrose-loaded Porcine Carrier Erythrocytes,” Am. J. Vet. Res. 44:1159-1161 (1983); DeLoach, J R, et al., “Preparation of Resealed Carrier Erythrocytes and In Vivo Survival in Dogs,” Am. J. Vet. Res. 42:667-669 (1981); Leung, P, et al., “Encapsulation of Thiosulfate: Cyanide Sulfurtransferase by Mouse Erythrocytes,” Toxicol. App. Pharm. 83:101-107 (1986); DeLoach, J R, et al., “A Dialysis Procedure for Loading Erythrocytes with Enzymes and Lipids,” Biochem. Biophys. Acta 496: 136-145 (1977); and Eichler, H G, et al., “In vivo clearance of antibody-sensitized human drug carrier erythrocytes,” Clin. Pharmacol. Ther. 40:300-303 (1986). Hypotonic dialysis can be performed on large quantities of red blood cells by use of automated apparatus or instrumentation. Examples are described by DeLoach et al., U.S. Pat. No. 4,327,710 (Mar. 4, 1982); Magnani et al., U.S. Pat. No. 6,139,836 (Oct. 31, 2000); and McHale, U.S. Pat. No. 6,495,351 B2 (Dec. 17, 2002).

The concentration of HbA1c in the hypotonic solution can vary depending on the target HbA1c concentration in the resulting cellular quality control. The target concentration itself can vary and is not critical to the control preparation procedure itself. In certain embodiments, the target concentration is one within the range of from about 1% to about 5%, and in others within the range of from about 5% to about 20%, all by weight.

Once loaded with HbA1c, the RBCs are de-permeabilized, i.e., their membranes are sealed against further migration of hemoglobin, whether glycated or non-glycated, across the membranes. De-permeabilization can be accomplished by conventional techniques known in the art. One method is gentle heating of the RBCs in the presence of a physiological solution, examples of which are phosphate-buffered saline and Ringer's solution. Another method is dialysis against a hypertonic solution, examples of which are disclosed in the references cited above. One example of a hypertonic solution is a solution containing 450 mM NaCl, 10 mM Na₂HPO₄, and 10 mM NaH₂PO₄ at pH 7.3 and osmolality greater than 850 mOsm/kg. Another example is a solution of 5 mM adenine, 100 mM inosine, 2 mM ATP, 100 mM glucose, 100 mM sodium pyruvate, 4 mM MgCl₂, 194 mM NaCl, 1.6 M KCl, and 35 mM NaH₂PO₄, pH 7.4 at a temperature of 37° C. for 20-30 minutes, or a solution of 100 mM phosphate (pH 8.0) and 150 mM NaCl at 25-50° C. for a period of time ranging from 30 minutes to four hours. Other solutions and methods will be readily apparent to those of skill in the art.

In embodiments that include the use of a fixing agent for the RBCs subsequent to the de-permeabilization, conventional fixing agents can be used. Examples are aliphatic dialdehydes, and in most cases those contain from 4-10 carbon atoms. Glutaraldehyde and paraformaldehyde are prominent examples. Other fixing agents can include, e.g., methanol and other alcohols, and acetone. Methods of fixation of the RBCs with the use of these fixing agents are known in the art.

Quality controls prepared in accordance with the procedures described above can be supplemented with conventional additives known for use in processed RBCs. Many such additives serving a variety of functions are known in the art and can be used. Included among these additives are stabilizers, of which magnesium gluconate, EDTA (ethylenediamine tetraacetic acid), and PEG (polyethyleneglycol) are examples. Further additives are antimicrobial agents, examples of which are neomycin sulfate, chloramphenicol, and sodium azide. Suitable concentrations of these additives will likewise be readily apparent to those of skill in the art. The additives can be applied to the RBCs during preliminary processing (i.e., prior to permeabilization), or during the permeabilization stage, the infusion stage, or the de-permeabilization stage, or two or more of these stages, by inclusion in the solution to which the cells are exposed. Alternatively or in addition, the additives can be included in a diluent in which the HbA1c-infused RBCs (i.e., RBCs containing HbA1c in encapsulated form) are suspended, when the HbA1c-infused RBCs are stored and used as a suspension. For quality controls in the form of suspensions, the osmolality of the suspension can vary but it will often be advantageous to maintain an osmolality that further contributes to the stabilization of the RBCs in the control. In such cases, the osmolality may range from about 200 to about 400 mOsm/kg. The composition of the final diluent can likewise vary, and in some cases the optimal composition may vary with the HbA1c level. Examples of components that can be included in the final diluent composition, often in any of several combinations, are magnesium gluconate, EDTA, PEG, sodium phosphate dibasic, glucose, methyl paraben, inosine, neomycin sulfate, chloramphenicol, potassium chloride, soybean trypsin inhibitor, sodium fluoride, ciprofloxacin, and sodium hydroxide.

RBCs treated in accordance with the procedures described above can be used by themselves as quality controls, or they can be blended with RBCs whose hemoglobin contents are unchanged from their original condition (i.e, their condition in the source from which they were originally obtained) in proportions that will result in averaged HbA1c concentrations that are at target levels that are intermediate to the two sets of RBCs. Thus, treatment of a single batch of RBCs can be used to prepare quality controls at two or more target levels by blending the infused and noninfused RBCs in different proportions. The choice of target levels can vary depending on the instrument on which the quality controls will be used, the assay whose accuracy will be monitored, and the disease condition sought to be detected or monitored.

Hematology assays and instruments on which the quality controls can be used include HemoPoint H2 and Hemoglobin A1c Test InView of Novo Nordisk (Princeton, N.J., USA), Hgb Pro Professional Hemoglobin Testing System of Spectrum Pharmaceuticals, Inc. (Henderson, Nev., USA), in2it™ A1C of Bio-Rad Laboratories, Inc. (Hercules, Calif., USA), DCA Vantage™ Analyzer of Siemens Healthcare Diagnostics (Tarrytown, N.Y., USA), and PDQ Plus™, PDQ Standalone, and ultra2™ A1c and Hemoglobin Variants Analyzers of Primus Corporation (Kansas City, Mo., USA).

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

What is claimed is:

1. A method of manufacturing a cellular hemoglobin A1c quality control comprising intact mammalian erythrocytes encapsulating hemoglobin A1c, said method comprising:
(a) dialyzing erythrocytes from a healthy mammal against a hypotonic solution under conditions causing permeabilization of cell membranes of said erythrocytes;
(b) contacting said erythrocytes having said permeabilized membranes with a solution of hemoglobin A1c at a selected concentration to infuse said erythrocytes with hemoglobin A1c from said solution; and
(c) contacting said erythrocytes so infused with a non-hypotonic solution under conditions causing de-permeabilization of said erythrocytes, thereby achieving intact erythrocytes with a stabilized level of encapsulated hemoglobin A1c.

2. The method of claim 1 wherein said selected concentration of hemoglobin A1c is from 1% to 5% by weight.

3. The method of claim 1 wherein said selected concentration of hemoglobin A1c is from 5% to 20% by weight.

4. The method of claim 1 further comprising fixing said erythrocytes subsequent to step (c) by treating said erythrocytes with an erythrocyte fixing agent.

5. The method of claim 1 wherein said non-hypotonic solution is a hypertonic solution.

6. The method of claim 1 further comprising combining said erythrocytes produced in step (c) with intact mammalian erythrocytes from a healthy mammal that have not undergone steps (a), (b), or (c) in a selected proportion to achieve a quality control with an intermediate level of hemoglobin A1c.

7. The method of claim 1 further comprising combining said erythrocytes produced in step (c) with intact mammalian erythrocytes from a healthy mammal that have not undergone steps (a), (b), or (c) in a plurality of proportions to achieve a plurality of quality controls at different levels of hemoglobin A1c.

8. A cellular hemoglobin A1c quality control prepared by the method of claim 1.

9. The cellular hemoglobin A1c quality control of claim 8 wherein said intact mammalian erythrocytes encapsulating hemoglobin A1c are suspended in a diluent having an osmolality of 200 to 400 mOsm/kg.

10. The cellular hemoglobin A1c quality control of claim 8 wherein said stabilized level of hemoglobin A1c is from 1% to 5% by weight.

11. The cellular hemoglobin A1c quality control of claim 8 wherein said stabilized level of hemoglobin A1c is from 5% to 20% by weight.
